(19) United States
(12) Patent Application Publication
(10) Pub. No.: US 2011/0296538 A1
(43) Pub. Date: Dec. 1, 2011

(54) IN VIVO QUANTITATIVE SCREENING TEST FOR ANTI-METASTASIS TREATMENT EFFICACY

(76) Inventors: Jeffrey Edward Segall, New Rochelle, NY (US); John Condeelis, City Island, NY (US); Dmitriy Kedrin, Bronx, NY (US); Jacco van Rheenen, Hilversum (NL); Bojana Gligorijevic, New York, NY (US)

(21) Appl. No.: 12/998,237

(22) PCT Filed: Oct. 28, 2009
(86) PCT No.: PCT/US2009/005851

§ 371 Date: Aug. 23, 2011

Related U.S. Application Data
(60) Provisional application No. 61/197,791, filed on Oct. 30, 2008.

(51) Int. Cl. A61K 49/00; C12N 5/09; A61P 35/00; A01K 67/027

(52) U.S. Cl. 800/3; 424/9.2; 800/18; 435/325

(57) ABSTRACT

The present invention is directed to methods for evaluating the efficacy of a cancer treatment for (i) inhibiting metastasis in a subject, (ii) inhibiting local cancer cell movement, and (iii) inhibiting cancer cell proliferation. The present invention is further directed to methods for monitoring cell motility in a subject. The present invention is also directed to kits for performing any of the above methods.

### CROSS-REFERENCE TO RELATED APPLICATION

This application claims priority of U.S. Provisional Patent Application No. 61/197,791, filed on Oct. 30, 2008, the content of which is incorporated by reference.

### STATEMENT OF GOVERNMENT SUPPORT

This invention was made with U.S. Government support under Grant Nos. CA100324 and CA77522 awarded by the National Cancer Institute, Grant Nos. U54GM64346 and U54CA126511 awarded by the National Institutes of Health, and Grant No. BC061403 awarded by the Department of Defense. The government has certain rights in this invention.

### FIELD OF THE INVENTION

The present invention generally relates to methods for evaluating the efficacy of a cancer treatment for inhibiting metastasis in a subject, methods for evaluating the efficacy of a cancer treatment for inhibiting cancer cell movement, methods for evaluating the efficacy of a cancer treatment for inhibiting cell proliferation, methods for monitoring cell motility, and related kits for these methods.

### BACKGROUND OF THE INVENTION

Throughout this application various publications are referred to by Arabic numerals in superscript. Full citations for these references may be found at the end of the specification immediately preceding the claims. The disclosures of these publications are hereby incorporated by reference in their entireties into the subject application to more fully describe the art to which the subject application pertains.

The early steps of metastasis are characterized by tumor cells invading the stroma (invasion) and entering the blood (intravasation)^{1,2}. Short term tracking of individual cells inside fluorescent tumors by intravital imaging has revealed dramatic heterogeneity in tumor cell invasion and intravasation³. However, long term tracking of individual cells is required to quantify these behaviors and to determine the fates of cells in specific tumor microenvironments. Imaging techniques that rely on surgical dissection to expose the imaging site have limitations for long-term experiments such (1) tissue dehydration, impaired thermoregulatory control and/or animal survival upon surgical dissection, (2) possible effects of prolonged anesthesia exposure, and (3) a limited field of view. These limitations can be overcome by studying tumors through a dorsal skinfold chamber⁴. The use of dorsal skinfold chambers, however, limits the experiments to tumor-models based on cell lines, and for many tumors a non-orthotopic environment. For example, invasion and intravasation of breast tumor cells is highly dependent on the specific local microenvironment⁵ which may not exist in the non-mammary environments such as the dorsal skinfold chamber site⁴. Thus, there is a need for improved methods for tracking cell movement, especially for tumor cells, in orthotic environments.

### SUMMARY OF THE INVENTION

The present inventors have discovered a new method for observing tumor cell metastasis in a subject using cancer cells transfected with photoswitchable proteins and an imaging window inserted into the subject. The present method avoids many of the drawbacks observed in previously used methods, e.g. skin flap dissection. The present method further allows for (i) high resolution in vivo imaging, (ii) the ability to image the same site over multiple days, and (iii) the ability to follow specifically marked subpopulations of cells.

The present invention is directed to methods for evaluating the efficacy of a potential cancer treatment for inhibiting metastasis in a subject comprising (a) inserting cancer cells transfected with a photoswitchable protein into a subject or obtaining a transgenic mouse subject in which a photoswitchable protein is expressed in cancer cells in the subject, (b) inserting an imaging window into the subject over the cancer cells, (c) photoswitching a cancer cell or cells, (d) administering the potential cancer treatment to the subject; and (e) observing the movement of the photoswitched cell or cells in the subject, wherein less movement of the photoswitched cell or cells in the subject in comparison to a similar subject not having been administered the cancer treatment indicates that the cancer treatment is effective in inhibiting metastasis, or wherein movement of the photoswitched cell or cells in the subject that is not significantly less in comparison to a similar subject not having been administered the cancer treatment indicates that the cancer treatment is ineffective for inhibiting metastasis.

The present invention is further directed to methods for evaluating the efficacy of a potential cancer treatment for inhibiting local cancer cell movement in a subject comprising (a) inserting cancer cells transfected with a photoswitchable protein into a subject or obtaining a transgenic mouse subject in which a photoswitchable protein is expressed in cancer cells in the subject, (b) inserting an imaging window into the subject to allow observation of the cancer cells, (c) photoswitching the photoswitchable protein expressed in one or more cancer cells, (d) administering the potential cancer treatment to the subject, and (e) observing the local movement of the photoswitched cell or cells over a period of time in the subject, wherein less movement of the photoswitched cell or cells in the subject in comparison to a similar subject not having been administered the cancer treatment indicates that the cancer treatment is effective in inhibiting local cancer cell movement, or wherein movement of the photoswitched cell or cells in the subject that is not significantly less in comparison to a similar subject not having been administered the cancer treatment indicates that the cancer treatment is ineffective for inhibiting local cell movement.

The present invention is further directed to methods for evaluating the efficacy of a potential cancer treatment for inhibiting cancer cell proliferation in a subject comprising, (a) inserting cancer cells transfected with a photoswitchable protein into a subject or obtaining a transgenic mouse subject in which a photoswitchable protein is expressed in cancer cells in the subject, (b) inserting an imaging window into the subject to allow observation of the cancer cells, (c) photoswitching the photoswitchable protein expressed in one or more cancer cells, (d) administering the potential cancer treatment to the subject, and (e) observing any increase in the number of photoswitched cells over a period of time in the subject, wherein a lower increase in the number of photoswitched cells in the subject in comparison to a similar subject not having been administered the cancer treatment indicates that the cancer treatment is effective in inhibiting cancer cell proliferation, or wherein an increase in the number of photoswitched cells in the subject that is not significantly less in comparison to a similar subject not having been administered the cancer treatment indicates that the cancer treatment is ineffective for inhibiting cancer cell proliferation.

The present invention is further directed to a kit for performing any of the above-described methods comprising two or more of the following: (a) a plasmid encoding a photoswitchable protein, (b) a cancer cell line, (c) one or more imaging windows, (d) an imaging box, and (e) instructions for photoswitching proteins, imaging cancer cells, and use of the imaging box.

The present invention is further directed to methods for monitoring cell motility in a subject comprising (a) inserting cells transfected with a photoswitchable protein into a subject or obtaining a transgenic mouse subject expressing a photoswitchable protein, (b) inserting an imaging window into the subject to allow observation of the cells, (c) photoswitching the photoswitchable protein expressed in one or more cells, and (d) observing the movement of the photoswitched cell or cells over a period of time in the subject, thereby monitoring cell motility in the subject.

The invention further provides a transgenic mouse subject in which a photoswitchable protein is expressed in cells of interest, such as cancer cells.

### BRIEF DESCRIPTION OF THE FIGURES

FIGS. 1a-1e. The Mammary Imaging Window (MIW) allows for long-term, high resolution imaging of the orthotopic tumors. (a) Components and the assembly of the MIW: a coverslip is mounted on a plastic frame consisting of two plastic rings and surgically implanted on top of the mammary gland or mammary tumor. (b,c) Average increase and decrease in signal for Dendra2, as measured in a region of interest, in cells in vitro (b) or in vivo (c) upon photoswitching. The values were normalized to the highest fluorescent level in red and the initial fluorescent level in green. The insets in both FIGS. 1b and 1c show the ratio of the non-normalized red and green fluorescence. (d) Cells within Dendra2-tumors were photoswitched through the MIW and the red fluorescence was quantified before, immediately after (0 days) photoswitching and for the 5 subsequent days. The values were normalized to the red fluorescence level before photoswitching. (e) Photoswitching of Dendra2-expressing MTLn3 tumor cells in vivo can be done in regions of interest ranging from one cell (left panel, scale bar 10 μm) to hundreds of cells (right panel, scale bar 75 μm) through the MIW. Shown are combined images from the green and red channels using an OR-function: Only the pixels in the red channel that are above background are shown, and for all other pixels, the green channel is shown.

FIGS. 2a-2f. Photoswitching through the MIW is a tool for studying orthotopic tumor microenvironments. (a) By using Dendra2 as the label for tumor cells (top left and right panels), Texas Red dextran for blood vessels (bottom right panel) and reflectance (bottom left panel) for extracellular matrix (ECM), one can define vascular microenvironments and monitor chosen cells inside them. (b,c) Non-photoswitched cells (lighter background cells) and photoswitched cells (darker cells in square-shaped area) are shown at 0 h, 6 h and 24 h after the photoswitch in avascular (b) and vascular (c) microenvironments (visible vessel indicated by white dotted lines). Shown are combined images from the green and red channels using an OR-function: Only the pixels in the red channel that are above background are shown, and for all other pixels, the green channel is shown. Scale bars 30 μm. The relative infiltration areas (d) and numbers (e) of photoswitched cells over time in vascular and avascular microenvironments. Error bars represent s.e.m., asterisk represents P<0.05, and n>20. (f) Detection of photoswitched cells in the lung. Lungs of an animal which had a large vascular area (20-40 mm²) of the primary tumor photoswitched were examined ex vivo 24h after photoswitching in green and red channels by epifluorescence microscopy. Arrow points to a red tumor cell photoswitched in and disseminated from the primary tumor. It was determined 0.009+/−0.007 (s.e.m.) red cells and 1.4+/−0.33 green cells per mm² lung, resulting in a green/red ratio of 152+/−0.81. Scale bar 20 μm.

FIGS. 3a-3d. Imaging box setup. View from the side (a) or the bottom (b) of the imaging box that was used to anesthetize and then immobilize the mouse for imaging. (c) For imaging Z-stacks, the imaging window was securely immobilized between two doors in the bottom of the box. (d) A flow of isoflurane enters the imaging box through a nozzle, and due to the negative pressure, the isoflurane exits the box passing through a carbon filter.

FIGS. 4a-4b. Comparative histology of Dendra2-MTLn3 tumors with and without MIW implant. (a) (I-III) control tumors without window, (IV-VI) tumors with MIW implant; (I, IV) H&E sections, scale bar 200 μm; (II, V) blood vessel staining using CD34 antibody; (III, VI) macrophages stained using F4/80, scale bar 50 μm. Tumors were dissected from the connective tissue and the mammary fat; the surfaces away from the MIW (or away from the skin) were marked with green tissue dye for orientation. In the sections shown, lines mark tumor areas where the MIW was located (IV-VI) or corresponding areas next to the skin in control tumors (I-III). Tumors were formalin-fixed and stained using H&E to visualize necrotic areas, general morphology, mitosis and apoptotic cells; CD34 antibody to visualize angiogenesis and F4/80 antibody to visualize possible inflammation. (b) Change in tumor volume due to MIW insertion. At day 0, before MIW insertion, tumor size was measured through the skin and animals with equal tumor sizes were marked as control-MIW pairs, with one member of each pair receiving a MIW and the other member of the pair left as the control. At 1, 4, 7 or 9 days after the MIW insertion, both members of the control-MIW pair were euthanized, the tumors removed and dimensions were measured. The ratio without MIW to with MIW was calculated for tumor volume. Regression analysis indicates no significant change in relative tumor volume with time (slope=−0.011, S.E. of slope=0.029, p<0.73) and no significant change in relative tumor volume of tumors analyzed at day 4, 7 or 9 compared to 1.

FIGS. 5a-5d. Quantification of invasion and intravasion of adenocarcinoma cells in different tumor microenvironments. (a) Visualization of multiple channels during intravital photoswitching of a single Dendra2-MTLn3 cell. Green and red channels represent Dendra2 before and after photoswitching, while purple denotes extracellular matrix visualized (ECM) via light scattering. Combined channel shows green, red and purple/ECM channels combined. (b) Diagram of three regions (250×250 μm) within the same orthotopic tumor that were photoswitched. Black areas represent visible blood vessels, while the square areas represent the sites of photoswitching. (c) Z-stacks of these regions were acquired at 0, 6 and 24 hours after photoswitching. (d) Single z-sections are shown. Maximum projections of the full z-stacks were made and the infiltration area (the area in which photoswitched cells can be found as shown with the light solid line) and numbers of photoswitched cells were measured over time.

FIGS. 6a-6b. Genetically encoded and transiently transfected tumor models can be visualized through the MIW. (a) MTLn3 cells that stably express cytosolic CFP were transiently transfected with a membrane targeted GFP (GFP-CAAX), and injected into the mammary fat pad. Several days after inoculation, high resolution CFP (central light area) and GFP (light outline around CFP) images were acquired through the MIW (scale bar 5 μm). (b) The MIW was inserted on top of a MMTV-PyMT GFP tumor. Images were acquired of tumor cells (light area) and the ECM (e.g., in particular bottom right area in 6b). Arrowheads indicate the interactions between the cells and the matrix fibers (scale bar 10 μm).

### DETAILED DESCRIPTION OF THE INVENTION

The present invention provides a method for evaluating the efficacy of a potential cancer treatment for inhibiting metastasis in a subject comprising (a) inserting cancer cells transfected with a photoswitchable protein into a subject or obtaining a transgenic mouse subject in which a photoswitchable protein is expressed in cancer cells in the subject, (b) inserting an imaging window into the subject over the cancer cells, (c) photoswitching a cancer cell or cells, (d) administering the potential cancer treatment to the subject, and (e) observing the movement (e.g., invasion and/or metastasis) of the photoswitched cell or cells in the subject, wherein less movement (e.g., invasion and/or metastasis) of the photoswitched cell or cells in the subject in comparison to a similar subject not having been administered the cancer treatment indicates that the cancer treatment is effective in inhibiting metastasis, or wherein movement (e.g., invasion and/or metastasis) of the photoswitched cell or cells in the subject that is not significantly less in comparison to a similar subject not having been administered the cancer treatment indicates that the cancer treatment is ineffective for inhibiting metastasis.

As used herein, “potential cancer treatment” shall mean any treatment with a possibility for inhibiting tumor metastasis in a subject. Types of cancer for which treatments can be evaluated by the present method include, but are not limited to, breast, pancreatic, gastric, ovarian, mesenteric, glandular, lung, rectal, stomach, bladder, head and neck, skin (e.g., melanoma) and brain (e.g., glioma) cancer. Additionally, any cancer cell line from any of the above cancer types can be transfected with the photoswitchable protein and used in the present method. In one embodiment, the cancer cell is the breast cancer cell MTLn3 or a carcinoma or glioblastoma or melanoma cell.

In the present method, the subject can be a mammal. In different embodiments, the mammal is, but not limited to, a mouse, a rat, a cat, a dog, a horse, a sheep, a cow, a steer, a bull, livestock, a primate, or a monkey. Preferably, the subject is a non-human animal model.

As used herein, “metastasis” shall mean the spread of cancer from one organ or location in the subject to another organ or part of the body. A cancer treatment that has efficacy in “inhibiting” metastasis shall mean a treatment that reduces the spread of cancer cells to areas in the subject beyond the initial tumor relative to the spread of cancer cells in a similar subject in the absence of the treatment. Conversely, a cancer treatment is ineffective in inhibiting metastasis if that treatment results in the movement of cancer cells that is not significantly less than the movement of cancer cells in a similar subject in the absence of the treatment.

As used herein, a “photoswitchable protein” shall mean any fluorescent protein whose fluorescent state can be modified. Preferably, the modification of the photoswitchable protein is facilitated by irradiation of the protein at a specific wavelength. Examples of photoswitchable proteins include, but are not limited to Dendra2, PS-CFP, PS-CFP2, and mOrange. In a preferred embodiment, the photoswitchable protein is Dendra2. Dendra2 and PSCFP are activated by light at approximately 405 nm. mOrange is activated using light at approximately 488 nm.

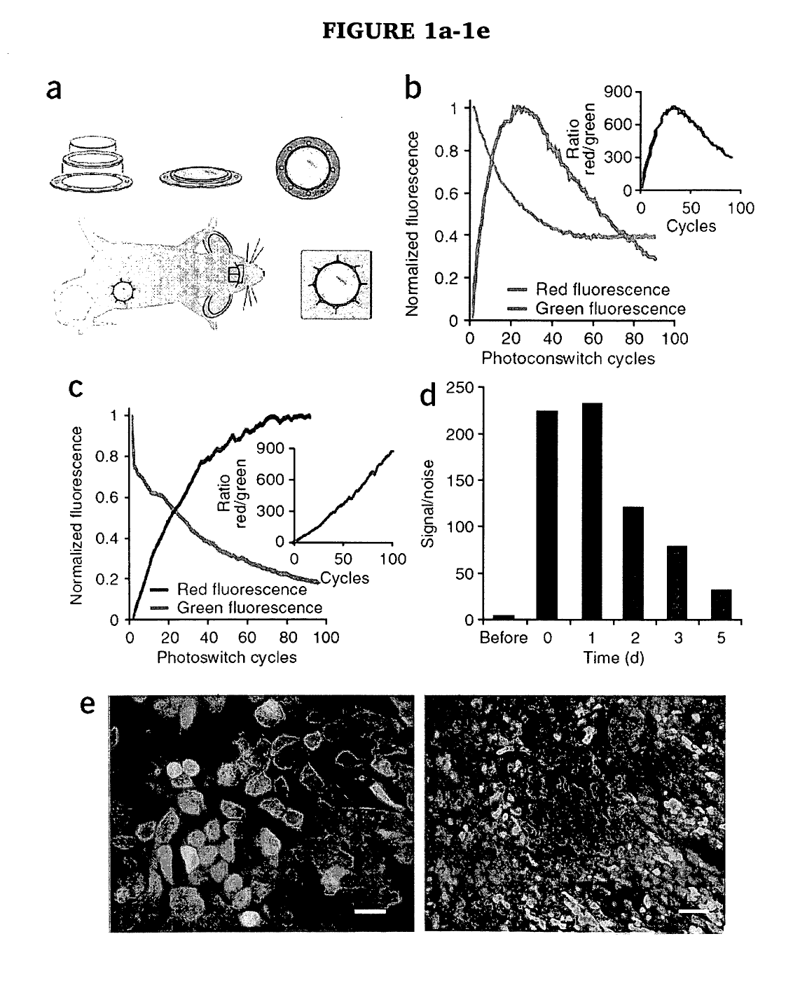

As used herein, an “imaging window” shall mean any apparatus inserted or transplanted into a subject comprising a transparent material, thus allowing observation of the interior of the subject. One example of an imaging window is shown in FIG. 1a.

An example of an antimetastasis assay can involve photoconverting cells in the primary tumor and then counting the number of photoswitched (e.g., red) cells that appear in the lungs or other metastatic sites, including bone marrow, liver, and brain. Treatments can be evaluated that reduce the number of (e.g., red) photoswitched cells that then get to the lungs in a defined period of time after photoswitching.

The present invention further provides a method for evaluating the efficacy of a potential cancer treatment for inhibiting local cancer cell movement in a subject comprising (a) inserting cancer cells transfected with a photoswitchable protein into a subject or obtaining a transgenic mouse subject in which a photoswitchable protein is expressed in cancer cells in the subject, (b) inserting an imaging window into the subject to allow observation of the cancer cells, (c) photoswitching the photoswitchable protein expressed in one or more cancer cells, (d) administering the potential cancer treatment to the subject, and (e) observing the local movement of the photoswitched cell or cells over a period of time in the subject, wherein less movement of the photoswitched cell or cells in the subject in comparison to a similar subject not having been administered the cancer treatment indicates that the cancer treatment is effective in inhibiting local cancer cell movement, or wherein movement of the photoswitched cell or cells in the subject that is not significantly less in comparison to a similar subject not having been administered the cancer treatment indicates that the cancer treatment is ineffective for inhibiting local cell movement.

As used herein, “local cancer cell movement” shall mean the spread of cancer cells from the initial tumor into the adjacent tissue. A cancer treatment that has efficacy in “inhibiting” local cancer cell movement shall mean a treatment that reduces the spread of cancer cells into the areas adjacent to the tumor in the subject relative to the spread of cancer cells in a similar subject in the absence of the treatment. Conversely, a cancer treatment is ineffective in inhibiting local cancer cell movement if that treatment results in the spread of cancer cells that is not significantly less than the spread of cancer cells in a similar subject in the absence of the treatment.

Photoswitchable proteins, cancer cells, subjects, and imaging windows suitable for use in this method are described above. In the preferred embodiment, the photoswitchable protein is Dendra2, mOrange or PSCFP.

The present invention further provides a method for evaluating the efficacy of a potential cancer treatment for inhibiting cancer cell proliferation in a subject comprising (a) inserting cancer cells transfected with a photoswitchable protein into a subject or obtaining a transgenic mouse subject in which a photoswitchable protein is expressed in cancer cells in the subject, (b) inserting an imaging window into the subject to allow observation of the cancer cells, (c) photoswitching the photoswitchable protein expressed in one or more cancer cells, (d) administering the potential cancer treatment to the subject, and (e) determining any increase in the number of photoswitched cells over a period of time in the subject, wherein a lower increase in the number of photoswitched cells in the subject in comparison to a similar subject not having been administered the cancer treatment indicates that the cancer treatment is effective in inhibiting cancer cell proliferation, or wherein an increase in the number of photoswitched cells in the subject that is not significantly less in comparison to a similar subject not having been administered the cancer treatment indicates that the cancer treatment is ineffective for inhibiting cancer cell proliferation.

As used herein, “cancer cell proliferation” shall mean an increase in the number of cancer cells. A cancer treatment that has efficacy in “inhibiting” cancer cell proliferation shall mean a treatment that results in a lower increase in the number of cancer cells relative to the increase observed in a similar subject in the absence of the treatment. Conversely, a cancer treatment is ineffective in inhibiting cancer cell proliferation if that treatment results in an increase in the number of cancer cells that is not significantly less that the increase observed in a similar subject in the absence of the treatment.

Photoswitchable proteins, cancer cells, subjects, and imaging windows suitable for use in this method are described above. In the preferred embodiment, the photoswitchable protein is Dendra2, mOrange or PSCFP.

The present invention further provides a kit for performing any of the above-described methods comprising two or more of the following: (a) a plasmid encoding a photoswitchable protein, (b) a cancer cell line, (c) one or more imaging windows, (d) an imaging box, and (e) instructions for photoswitching, imaging of the cancer cells, and use of the imaging box. In another embodiment, the kit comprises three or more of the above-described components.

Plasmids are well-known in the art and one skilled in the art would be able to produce a plasmid encoding a photoswitchable protein such that the protein can be expressed in a cell without undue experimentation.

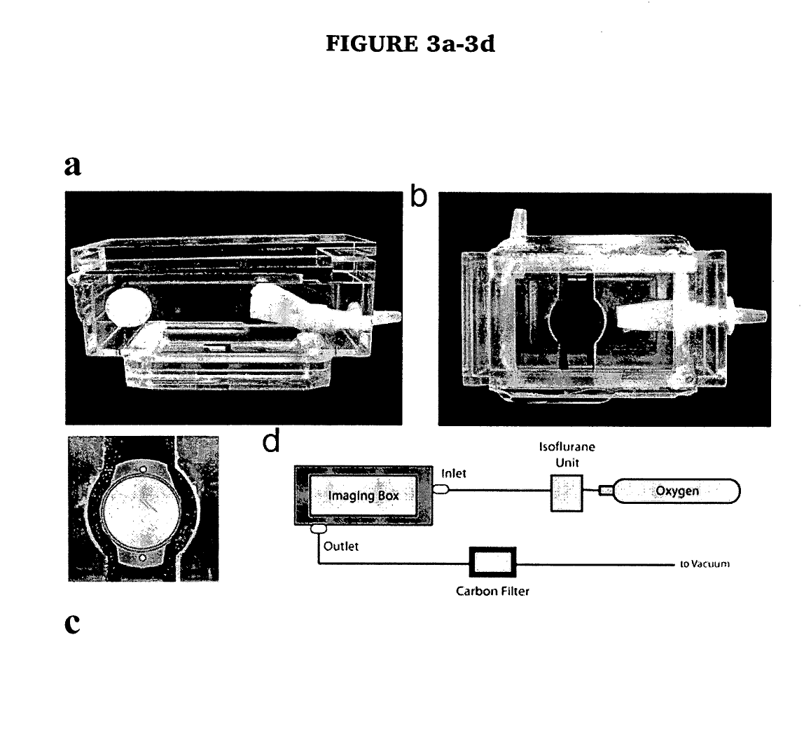

As used herein, an “imaging box” shall mean any apparatus to aid in securing and observing the subject having an imaging window inserted therein. On example of an imaging box is shown in FIGS. 3a-3d.

Additionally, any of the above-described cancer cell lines, photoswitchable proteins, and imaging windows are suitable components for the present kit.

The present invention further provides a method for monitoring cell motility in a subject comprising (a) inserting cells transfected with a photoswitchable protein into a subject or obtaining a transgenic mouse subject in which a photoswitchable protein is expressed in cells of interest in the subject, (b) inserting an imaging window into the subject to allow observation of the cells, (c) photoswitching the photoswitchable protein expressed in one or more cells, and (d) observing the movement of the photoswitched cell or cells over a period of time in the subject, thereby monitoring cell motility in the subject.

Any of the above described photoswitchable proteins and subjects are suitable for use in the present method. In the preferred embodiment, the photoswitchable protein is Dendra2, mOrange or PSCFP.

The present invention further provides a kit for monitoring cell motility in a subject comprising two or more of the following: (a) a plasmid encoding a photoswitchable protein, (b) a cell line, (c) one or more imaging windows; (d) an imaging box, and (e) instructions for photoswitching, imaging of the cells, and use of the imaging box. In another embodiment, the kit comprises three or more of the above described components.

Additionally, any of the above described photoswitchable proteins, imaging windows, and imaging boxes are suitable components for the present kit. In the preferred embodiment, the photoswitchable protein is Dendra2.

The invention further provides a transgenic mouse subject in which a photoswitchable protein is expressed in cells of interest, such as cancer cells. Such transgenic mice can be produced by expressing a photoswitchable protein (such as Dendra2) in either multiple tissues in a mouse or in a specific tissue, using a promoter driving Dendra2 gene expression. In addition, a tumor can then be induced, either using a promoter to drive expression of an oncogene in tissues that express Dendra2, or else using other treatments (such as application of chemicals) to induce tumor formation. Then, once the tumor is formed, the MIW can be inserted, and the cells in the tumor can be photoconverted and tracked.

The methods disclosed herein can be further used for evaluation at the level of cellular resolution, for example changes in protein localization. The methods also provide for distinction of invasion, intravasation and dissemination at the single cell level. The methods further provide for analysis of proliferation and self renewal of small groups of cells, e.g., cancer stem cells.

This invention will be better understood from the Experimental Details, which follow. However, one skilled in the art will readily appreciate that the specific methods and results discussed are merely illustrative of the invention as described more fully in the claims that follow thereafter.

### Experimental Details

### Materials and Methods

Cell Culture. MTLn3 cells, originally isolated by Neri and Nicolson (Institute for Molecular Medicine, Huntington Beach, Calif.), were maintained in αMEM (Life Technologies, Inc., Gaithersburg, Md.) with 5% FBS and penicillin-streptomycin (Life Technologies, Inc.). Dendra2 was in the cloning vector C1 with the G418 selection marker. Transfection was done using Lipofectamine 2000 (Invitrogen, CA). Sorting of the cells was done using FACS 72 h after the transfection; 4% of the cells expressing the highest fluorescence levels were kept for culturing and selection was maintained using 500 μg/ml G418 geneticin (Invitrogen, CA). No changes in cell morphology, viability or proliferation in Dendra2-MTLn3 cells compared to GFP-MTLn3 cells were observed.

Choice of the Photoswitchable Protein. Photoswitchable fluorescent proteins represent a group of fluorescent proteins which all exhibit high level of amino acid identity with EGFP; they are genetically encoded probes which allow instant labeling of proteins, organelles or cells with light of a specific wavelength. In contrast to so called photoactivatable proteins, such as photoactivatable GFP, which do not fluoresce before photoconversion (i.e., are dark), the photoswitchable proteins are already fluorescent in one spectral range and then change the fluorescence spectrum after a brief light irradiation. Currently, there are two types of the protein that are photoswitching either from cyan to green or from green to red. Since cellular and tissue autofluorescence tends to decrease with increasing wavelength, the latter type was preferred. Also, among the green-to-red phototoswitchable proteins there are monomeric as well as tetrameric proteins. Tetrameric fluorescent proteins such as DsRed are known to frequently cause formation of intracellular aggregates and a significant decrease in viability which complicates establishing of stable clones. This is why it was decided to use the monomeric green-to-red protein, Dendra2. Additional advantages of Dendra2 over the other monomeric green-to-red proteins are that it completely forms a functional chromophore at 37° C., which is not the case for mEosFP, and it is substantially brighter than the original Dendra protein.

Design of the Imaging Window. Design of the imaging window Mammary Imaging Window (MIW) base is made from tissue culture-grade plastic dishes (Becton Dickinson #353004) by cutting out ring-shaped pieces. Dimensions of the smaller ring are 9 mm for internal and 10 mm for external diameter, 9 mm/14 mm for larger ring. This design assures MIW touches the mammary gland surface while allowing for suturing and proper window positioning inside the imaging box. Both parts are sanded and glued together. In order to secure the window in the animal, 8 suturing holes are made on the larger piece. Holes were equally positioned around the edge of the smaller ring. The coverslip (8 mm, #1, circular, custom made by Fisher Scientific) was attached to the top of the small ring using superglue. Further, MIW was rinsed with sterile water and ethanol, dried and placed under the UV light in the tissue culture dish for at least 12 h on each side. Rather than the ring system described above, molds have also been designed and used where the molds form MIW plastic shapes to which a coverslip is then glued.

Window Insertion Surgery. Female 5- to 6-week-old BALB/c SCID/Ncr mice were purchased from the National Cancer Institute and housed in a pathogen-free barrier facility until use. Mice were first injected with 10⁶ MTLn3 cells in the abdominal mammary fat pad. After 15-17 days, when the tumor reached 0.5 cm in diameter, the window was inserted under the skin on top of the tumor. Surgeries were done in the quarantine room according to the survival surgery protocol approved by AECOM Animal Institute. A sterile cloth is placed inside a laminar flow hood, with a heating pad (36° C.) under it. Sterile dissecting scissors, tweezers, cotton swabs and suturing thread were placed on the cloth. Mice were anesthetized with intraperitoneal injection of 0.35 ml of Tribromo-ethanol (Avertin 2%) and placed on a piece of gauze. The tumor surface was shaved and the rest of the hair was removed with lotion hair remover. The skin was disinfected with 70% EtOH and Betadine, and the mouse was transferred on the sterile cloth. The skin dissection was started at the nipple area and no skin was cut away. A 5 mm long incision was made and the skin above the tumor was dissected away from the tumor using micro-dissecting scissors 3¼″ (BRI, Inc. #11-1080). Once enough of the surface of the tumor was detached from the skin, a sterile window was inserted under the skin and positioned so the glass touches the intact tumor. The skin was placed over the MIW edge and sutured through the holes to secure the MIW in place. Tissue glue (cyanoacrylate tissue adhesive) was used to seal the suturing holes and attach the window to the edges of the skin. The animal was allowed to recover and placed back into the cage. As a prophylaxis, TMP-SMX antibiotic (Hi-Tech Pharmacal #NDC 50383-824-16) is given by supplementing the water bottle with 3 ml of the antibiotic solution. Imaging was done 2-9 days after surgery.

Image Acquisition and Analysis. Isoflurane (Aerrane) was used to sedate the mice during short imaging sessions. The anesthetized animal was placed inside a custom box designed to maintain a temperature of 32 C.°. The box introduces isoflurane through a face mask and exchanges air through an inlet and outlet. The MIW was secured at the bottom of the box, with the coverslip exposed through an opening facing the objective of the microscope. A Leica TCS SP2 AOBS confocal microscope (Mannheim, Germany) was used for intravital imaging. The microscope is equipped with 63× (Leica HCX PL APO, NA 1.3, WD 0.6 mm) and 20× (HC PL APO NA 0.7, WD 1 mm) glycerol objectives. Similar results can be obtained with a multiphoton microscope with greater maximal depth of imaging. The argon laser 488 nm line was used at up to 10% for imaging of the green Dendra2 variant and ECM (averaging 60 μW through the objective). Photoswitching was done using full power of a blue diode laser emitting at 405 nm (average power 10 μW through the objective) with 4.9 μs dwell time per pixel. Excitation of the red Dendra2 variant was done using up to 15% power of a red diode laser emitting at 561 nm (averaging 30 μW at the objective), and excitation of AlexaFluor647-10K dextran using up to 20% HeNe laser power at 633 nm. Green Dendra2 variant fluorescence was collected between 510-560 nm; red Dendra2 fluorescence was collected between 580-620 nm, AlexaFluor647-10K dextran fluorescence was collected between 650-700 nm, and the ECM scattering was collected between 470-500 nm. Z-stacks were obtained up to a depth of 40-60 μm into the tissue. Multiphoton microscopy allowed for imaging ˜100 μm deep into the tissue. Multiphoton images of photoswitched Dendra were acquired with a BioRad Radiance 2000 Multiphoton Microscope using an inverted Olympus 1×70 microscope and a 40×0.9 NA. The two-photon microscope is equipped with 450/30 emission filter for second harmonic light, 536/40 for collection of Dendra2 emission pre-photoswitching and 593/40 for Dendra2 post-switching. Z-stack presented in Supplementary movie 3 was obtained using 860 nm excitation light delivered by Tsunami laser averaging 200 mW at the objective. The collected 512×512 pixels Z-stacks were stored and analyzed with Image J (Rasband, W. S., ImageJ, U.S. National Institutes of Health, Bethesda, Md., USA, http://rsb.info.nih.gov/ij/, 1997-2006). For migration analysis, a maximum projection was generated for the red photoswitched Z-stack, and the area in which photoswitched cells were found was measured, and normalized to the area at time 0. RGB images were processed as an OR function between Red and Green channels. The number of cells was counted in the z-stack, and multiple counts of the same cell in different slices were avoided by marking cells in each plane with a custom ImageJ plug-in. In dense areas, a size threshold was applied: each peak in fluorescence of maximum width<10 μm around the center was defined as a single cell. Results represented for Dendra2 MTLn3 cells were similar to those of Eos (tetrameric photoswitchable protein) in MTLn3 cells (total in vivo experiment N>20).

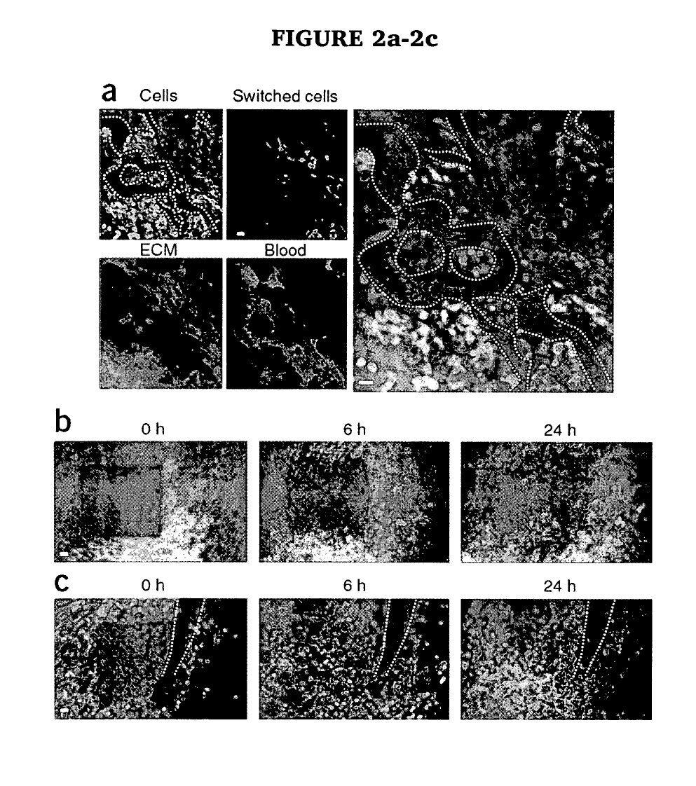

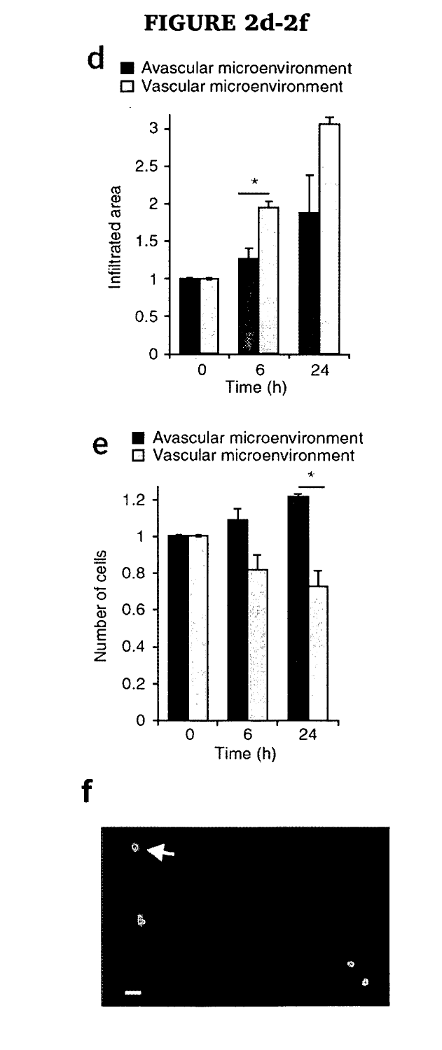

Equipment and Settings for Figures. FIG. 1: Images (512×512 pixels, 8 bits) of a Dendra2 MTLn3 tumor were acquired through the MIW using a Leica TCS SP2 AOBS confocal microscope (Mannheim, Germany) equipped with a 20× (HC PL APO NA 0.7, WD 1 mm) glycerol objectives at 32 C.°. Photoconversion was done with a 405 nm laser. For excitation of the green Dendra2 form, the argon laser 488 nm line was used, and for the red Dendra2 form a red diode laser emitting at 561 nm. Green Dendra2 form fluorescence was collected between 510-560 nm; red Dendra2 form fluorescence was collected between 580-620 nm. In FIG. 1e, the acquired images were processed with Image J using an OR function between Red and Green channel that works as follow: Only the pixels in the red channel that are above background are shown, and for all other pixels, the green channel is shown. FIG. 2: XYZ stacks (512×512 pixels, 8 bits) of a Dendra2 MTLn3 tumor were acquired through the MIW using a Leica TCS SP2 AOBS confocal microscope (Mannheim, Germany) equipped with a 20× (HC PL APO NA 0.7, WD 1 mm) glycerol objectives at 32 C.°. Photoconversion was done with a 405 nm laser. For excitation of the green Dendra2 form, the argon laser 488 nm line was used, and for the red Dendra 2 form a red diode laser emitting at 561 nm. Green Dendra2 form fluorescence was collected between 510-560 nm; red Dendra2 form fluorescence was collected between 580-620 nm. AlexaFluor647-10K dextran fluorescence upon 633 nm excitation was collected between 650-700 nm, and the ECM scattering upon 488 nm excitation was collected between 470-500 nm. All RGB images were generated in Image J. In FIG. 2a-c, the acquired images were processed with Image J. Three images of the Z-stack were averaged and a RGB image was generated using an OR function between Red and Green channel that works as follow: Only the pixels in the red channel that are above background are shown, and for all other pixels, the green channel is shown. For calculating the infiltration area, a maximum projection of the Z-stacks were generated in Image J, and the area in which photoswitched cells were found was measured, and normalized to the area at time 0.

Detection of Photoswitched Cells in the Lung. Lungs of an animal which had a large area (20 mm²) of the primary tumor photoswitched were examined ex vivo 24h after photoswitching in green and red channels by epifluorescence microscopy. In the green channel, only single green cells were taken into account. The average number of single green cells was 1.4+/−0.33 cells/mm² and average number of red cells was 0.009+/−0.007 cells/mm², resulting in green/red ratio of 152.

This number of cells is close to what can be expected theoretically: The photoswitched volume is 2 mm³ when photoswitched cells are positioned inside the window surface area (20 mm²) and a 0-0.1 mm depth of switching is assumed. Knowing that average tumor volume is 523 mm³ (average size measured at 4 days post-implantation of the MIW, data not shown)) the number of the tumor cells that can be photoswitched is 0.38% of total cells, only some of which will be adjacent to blood vessels. This means the ratio of green/red cells in the lungs should equal 0.9962/0.0038=262 in case all the red cells were in areas close to vessels, and larger if some of them were far from the vessels.

### Results

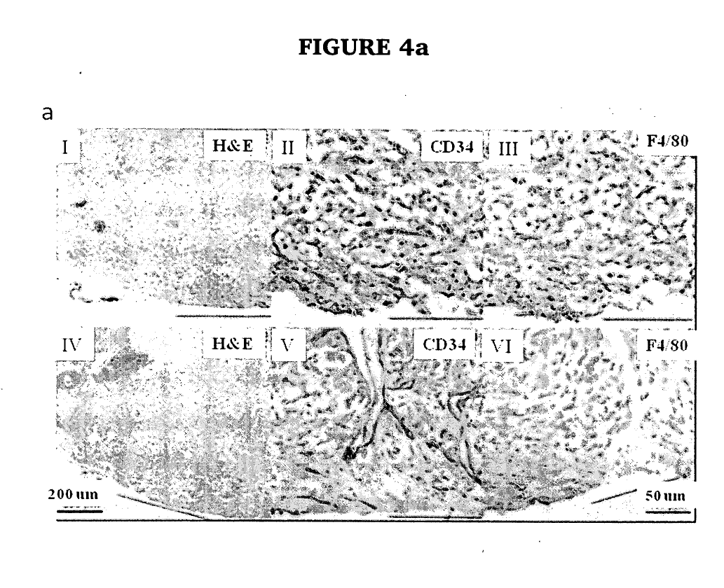

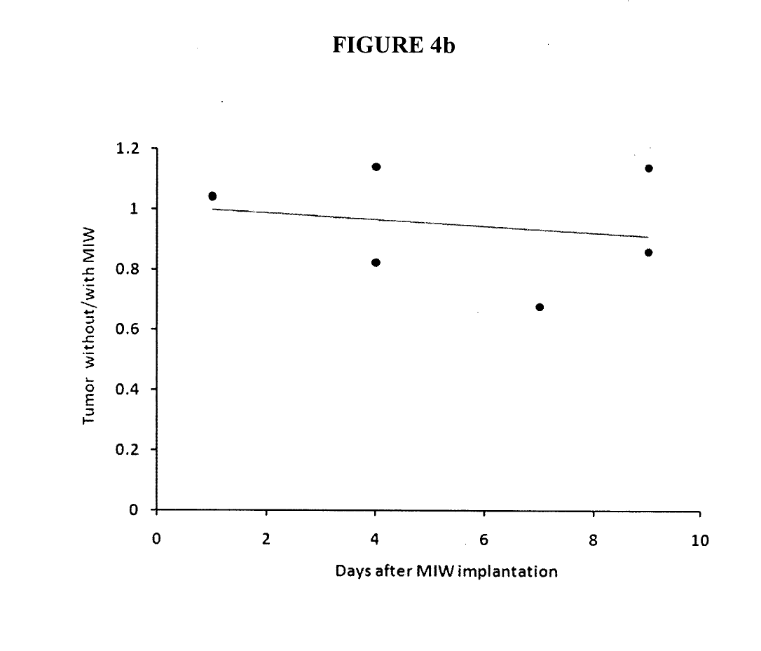

To image orthotopic (in the natural, mammary gland environment) breast tumors intravitally at high resolution for prolonged times, a MIW was developed that can be placed on top of the mammary gland of a mouse (FIG. 1a). The protocol for these animal studies was approved by the Institutional Animal Care and Use Committee for the Albert Einstein College of Medicine. The MIW consists of two plastic rings which form a mount for a glass coverslip. The mount has holes which facilitate suturing into the skin, whereas the glass coverslip assures the optimal working distance and refraction index for high resolution imaging (for more details on the equipment used for imaging, see FIG. 3). While surgical dissection of the skin overlaying the imaging site allows for several hours of data to be collected and is usually a terminal procedure, imaging through the MIW extended the imaging time to multiple days (up to 21 days). Tumors with MIW implants did not show inflammation, or a change in growth and microenvironments scored at 1-9 days after the implantation procedure (FIG. 4 and Table 1). In order to locate the same subpopulation of cells in each of the imaging sessions, reference points were required⁶. In the fast changing tissue topology of the tumor, the use of fixed reference points is limited, and therefore photoswitchable fluorescent proteins^{7,8} were used as photomarkers of the cells of interest. These proteins represent a new group of GFP-like fluorophores which allow labeling and tracking of a single cell or a group of cells^{9, 10, 11, 12}. The photoswitchable protein Dendra2 was stably expressed in the metastatic breast cancer line MTLn3. Dendra2 resembles GFP in its spectrum prior to photoswitching, but exposure to blue light (e.g. 405 nm) can induce an irreversible red shift>150 nm in the excitation and emission spectra of the chromophore¹³. Following the photoswitch, the red fluorescence stably increases up to 250 fold both in vitro and (FIG. 1b) and in vivo (FIG. 1c), resulting in red/green contrast of up to 850 and allowing us to track cells marked in this way. Five days after photoswitching, the red fluorescence of the photoswitched cells is still 31 fold higher than the red fluorescence of non-switched cells, which enables the recognition of the highlighted cells in vivo for extended times after the photoswitch (FIG. 1d).

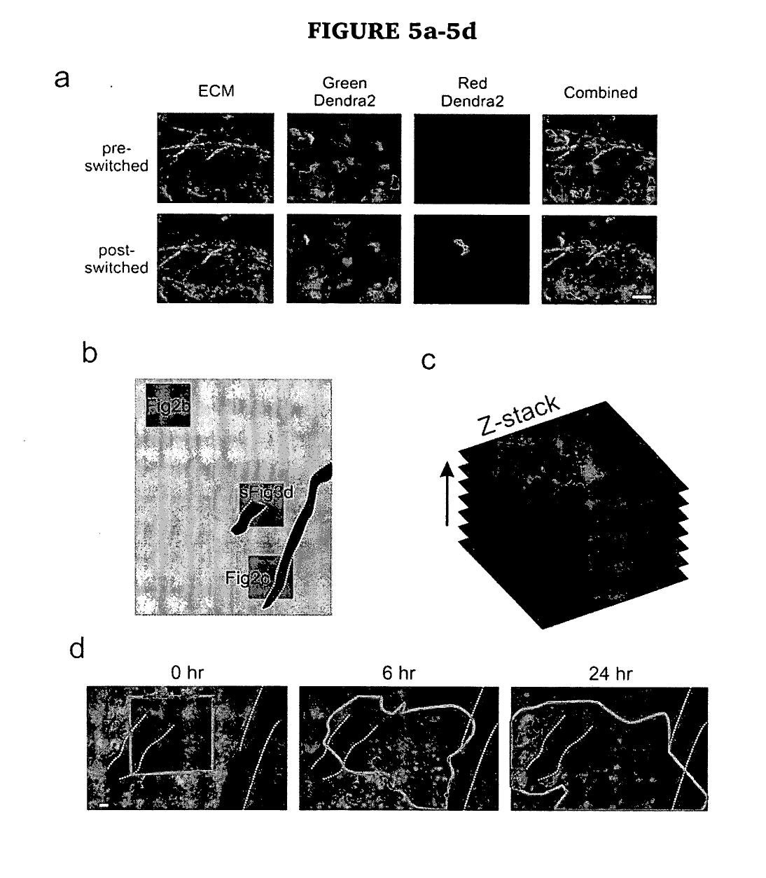

Regions containing one to hundreds of cells can be photoswitched and imaged through the MIW (FIG. 1e and FIG. 5). As cells in the tumor migrate and invade, the distribution of these cells relative to blood vessels and other tumor cells changed over time. By selectively photoswitching the fluorophore in a group of cells as demonstrated in FIG. 1e, the changes in distribution of cells in the tumor microenvironment was visualized. Twenty four hours after photo-switching, images of the non-photoswitched tumor cells (green), photoswitched cells (red), extracellular matrix (collagen was visualized with reflectance in FIG. 2a) and blood vessels (fluorescent dextrans were used for vessel labeling) were recorded. Interestingly, some photoswitched regions showed dramatic migration and invasion of the surrounding microenvironment (FIG. 2a).

The tumor perivascular microenvironment (tumor tissue surrounding blood vessels) is enriched in tumor-associated-macrophages and extracellular matrix, which supports metastatic behavior, including inhibition of proliferation and stimulation of migration, invasion and intravasation^{5,14}. This suggests the existence of distinct mammary tumor microenvironments within the same tumor with different rates of invasion and intravasation. However, the long term implications of these observations required the ability to revisit distinct microenvironments inside the same mammary gland over a 24-hour period. In order to quantify invasion and intravasation within distinct mammary gland microenvironments, photoswitched square regions (˜300 cells) were photoswitched in different tumor microenvironments of the same orthotopically grown tumor (FIG. 5), focusing on regions lacking (FIG. 2b) and containing (FIG. 2c) detectable blood vessels. The location of the photoswitched red cells was determined by acquiring Z-stacks of red and green images of the same regions at 0, 6 and 24 hours after photoswitching. In regions not containing detectable vessels (FIG. 2b) there was limited migration (FIG. 2d) and the number of cells increased (FIG. 2e), suggesting that this microenvironment does not support metastatic behavior. In contrast, cells in the vascular microenvironment (FIG. 2c) have infiltrated larger areas (FIG. 2d) and even migrated to sites outside of the field of view. Moreover, cells in this vascular microenvironment lined up along the blood vessel (FIG. 2c), with a concomitant decrease in the number of red tumor cells (FIG. 2e), and the appearance of red tumor cells in the lung (FIG. 2f). From these experiments, it was concluded that cell behavior is determined by the surrounding microenvironment, and that the vascular microenvironment promotes invasion and intravasation of tumor cells.

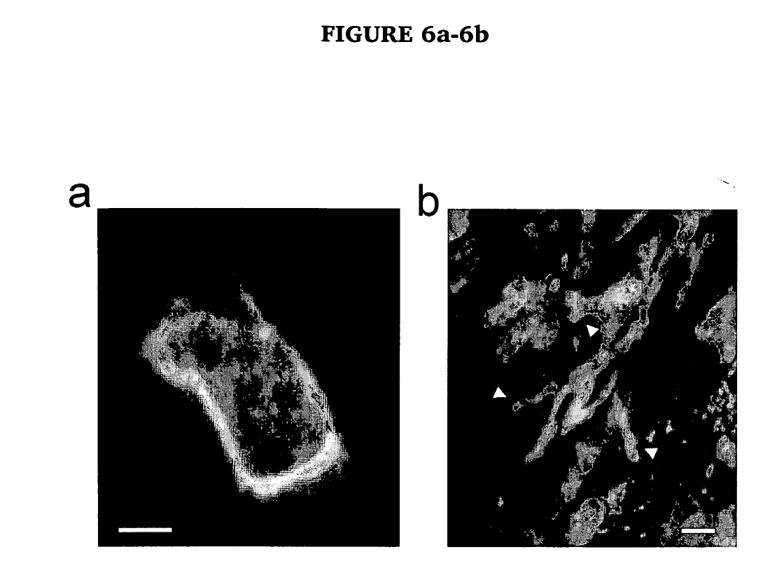

While the existence of different tumor microenvironments has been reported previously⁵, the quantitative analysis of such microenvironments in the same tumor with spatial and temporal resolution, is not possible with previous techniques. The combination of photoswitchable proteins with the MIW allowed for such analysis, since a distinct group of cells can be photomarked in any location in the primary tumor, and tracked over time without long term anesthesia. Furthermore, the high stability of Dendra2 enabled one to freeze fix the tissues and analyze them by microscopy without additional labeling. A limitation of Dendra2 is that a limited number of excitation wavelengths can be used. For example, since violet light causes a switch, DAPI stains should only be used after imaging other wavelengths as DAPI imaging would cause all green cells to then become red. Nevertheless, through the MIW, the formation of tumors from injection of cells in the mammary gland can be followed for days, which is not possible in surgically dissected areas. This also opens the possibility to study fluorescently-tagged proteins that have lethal effects if stably expressed, but can be studied in transiently transfected cells. For example, transiently transfected cells expressing membrane targeted GFP and injected into the mammary gland can be imaged with high resolution through the MIW (FIG. 6a). Although this would also be possible with the dorsal skinfold chamber⁴, imaging through the MIW allows studies of cell behavior in their physiological breast microenvironments and moreover, the MIW technology can be extended to tumors of transgenic origin, such as the MMTV-PyMT tumor model (FIG. 6b). Such tumor models allow the investigation of different stages of tumor progression¹⁵ in contrast to cell-line derived xenografts. The combined use of MIW and photomarking cells to revisit chosen subpopulations of cells is an important capability not only in tumor studies but any studies related to cell motility and morphogenesis. Visualization of infectious agents and immune responses, or the progression of chronic inflammation, are other examples of the potential applications of the technique described in this manuscript. This technique will also be helpful in monitoring of identification and proliferation of mammary stem cells, mammary gland growth and morphogenesis or testing artificial tissue heterotransplants.

TABLE 1
Quantification of histological sections.

| | | | Number of | Number of | Distance |
|---|---|---|---|---|---|
| | | Photo- | vessels/ | macrophages/ | edge-necrotic |
| Dendra 2 | MIW | switch | mm² | mm² | zone (mm) |
| − | − | − | 91 ± 3.0 | 10 ± 5.4 | 0.4 ± 0.24 |
| + | − | − | 53 ± 13.7 | 8 ± 3.0 | 1.3 ± 0.92 |
| + | + | − | 44 ± 4.2 | 10 ± 3.8 | 0.5 ± 0.45 |
| + | + | + | 48 ± 10.7 | 8 ± 3.2 | 0.8 ± 0.54 |

Tumors with and without Dendra2 transgene, MIW and photoswitching 24 h prior to fixation are compared in number of vessels (based on CD34 staining) and macrophage (based on F$/80 staining) and distance from the tumor edge to the closest necrotic zone. All the measurements were done in the areas covered by MIW or skin (in control tumors without MIW). T-tests show no statistical significance between populations.

### REFERENCES

1. Condeelis, J. and Segall, J. E., Intravital imaging of cell movement in tumours. Nat. Rev. Cancer 3 (12), 921 (2003).
2. Gupta, G. P. and Massague, J., Cancer metastasis: building a framework. Cell 127 (4), 679 (2006).
3. Sidani, M. et al., Probing the microenvironment of mammary tumors using multiphoton microscopy. J Mammary Gland Biol. Neoplasia 11 (2), 151 (2006).
4. Lehr, H. A. et al., Dorsal skinfold chamber technique for intravital microscopy in nude mice. The American journal of pathology 143 (4), 1055 (1993).
5. Wyckoff, J. B. et al., Direct visualization of macrophage-assisted tumor cell intravasation in mammary tumors. Cancer research 67 (6), 2649 (2007).
6. Bins, A. D. et al., Intravital imaging of fluorescent markers and FRET probes by DNA tattooing. BMC biotechnology 7, 2 (2007).
7. Gurskaya, N. G. et al., Engineering of a monomeric green-to-red photoactivatable fluorescent protein induced by blue light. Nat Biotechnol. 24 (4), 461 (2006).
8. Lukyanov, K. A., Chudakov, D. M., Lukyanov, S., and Verkhusha, V. V., Innovation: Photoactivatable fluorescent proteins. Nat. Rev. Mol. Cell Biol. 6 (11), 885 (2005).
9. Gray, N. W., Weimer, R. M., Bureau, I., and Svoboda, K, Rapid Redistribution of Synaptic PSD-95 in the Neocortex In Vivo. PloS Biol. 4 (11), e370 (2006).
10. Sato, T., Takahoko, M., and Okamoto, H., HuC:Kaede, a useful tool to label neural morphologies in networks in vivo. Genesis 44 (3), 136 (2006).
11. Hatta, K., Tsujii, H., and Omura, T., Cell tracking using a photoconvertible fluorescent protein. Nat. Protoc. 1 (2), 960 (2006).
12. Post, J. N., Lidke, K. A., Rieger, B., and Arndt-Jovin, D. J., One- and two-photon photoactivation of a paGFP-fusion protein in live Drosophila embryos. FEBS Lett. 579 (2), 325 (2005).
13. Chudakov, D. M., Lukyanov, S., and Lukyanov, K. A., Tracking intracellular protein movements using photoswitchable fluorescent proteins PS-CFP2 and Dendra2. Nature protocols 2 (8), 2024 (2007).
14. Condeelis, John and Pollard, Jeffrey W., Macrophages: Obligate Partners for Tumor Cell Migration, Invasion, and Metastasis. Cell 124 (2), 263 (2006).
15. Lin, E. Y. et al., Progression to malignancy in the polyoma middle T oncoprotein mouse breast cancer model provides a reliable model for human diseases. The American journal of pathology 163 (5), 2113 (2003).

1. A method for evaluating the efficacy of a potential cancer treatment for inhibiting metastasis in a subject, or for inhibiting local cancer cell movement in a subject, or for inhibiting cancer cell proliferation in a subject, the method comprising:
(a) inserting cancer cells transfected with a photoswitchable protein into a subject or obtaining a transgenic mouse subject in which a photoswitchable protein is expressed in cancer cells in the subject;
(b) inserting an imaging window into the subject to allow observation of the cancer cells;
(c) photoswitching the photoswitchable protein expressed in one or more cancer cells;
(d) administering the potential cancer treatment to the subject; and
(e) observing the spreading of the photoswitched cell or cells over a period of time in the subject, wherein less spreading of the photoswitched cell or cells in the subject in comparison to a similar subject not having been administered the cancer treatment indicates that the cancer treatment is effective in inhibiting metastasis, or wherein movement of the photoswitched cell or cells in the subject that is not significantly less in comparison to a similar subject not having been administered the cancer treatment indicates that the cancer treatment is ineffective for inhibiting metastasis; or
observing the local movement of the photoswitched cell or cells over a period of time in the subject, wherein less movement of the photoswitched cell or cells in the subject in comparison to a similar subject not having been administered the cancer treatment indicates that the cancer treatment is effective in inhibiting local cancer cell movement, or wherein movement of the photoswitched cell or cells in the subject that is not significantly less in comparison to a similar subject not having been administered the cancer treatment indicates that the cancer treatment is ineffective for inhibiting local cell movement; or
determining any increase in the number of photoswitched cells over a period of time in the subject, wherein a lower increase in the number of photoswitched cells in the subject in comparison to a similar subject not having been administered the cancer treatment indicates that the cancer treatment is effective in inhibiting cancer cell proliferation, or wherein an increase in the number of photoswitched cells in the subject that is not significantly less in comparison to a similar subject not having been administered the cancer treatment indicates that the cancer treatment is ineffective for inhibiting cancer cell proliferation.

2. The method of claim 1 for evaluating the efficacy of a potential cancer treatment for inhibiting local cancer cell movement in a subject comprising:
observing the local movement of the photoswitched cell or cells over a period of time in the subject, wherein less movement of the photoswitched cell or cells in the subject in comparison to a similar subject not having been administered the cancer treatment indicates that the cancer treatment is effective in inhibiting local cancer cell movement, or wherein movement of the photoswitched cell or cells in the subject that is not significantly less in comparison to a similar subject not having been administered the cancer treatment indicates that the cancer treatment is ineffective for inhibiting local cell movement.

3. The method of claim 1 for evaluating the efficacy of a potential cancer treatment for inhibiting cancer cell proliferation in a subject comprising:
determining any increase in the number of photoswitched cells over a period of time in the subject, wherein a lower increase in the number of photoswitched cells in the subject in comparison to a similar subject not having been administered the cancer treatment indicates that the cancer treatment is effective in inhibiting cancer cell proliferation, or wherein an increase in the number of photoswitched cells in the subject that is not significantly less in comparison to a similar subject not having been administered the cancer treatment indicates that the cancer treatment is ineffective for inhibiting cancer cell proliferation.

4. The method of claim 1, wherein the subject is a mouse or a rat.

5. The method of claim 1, wherein the cancer cells are breast cancer cells, glioma cells, head and neck cancer cells, or melanoma cells.

6. The method of claim 5, wherein the cancer cell is a carcinoma, glioblastoma or melanoma cell.

7. The method of claim 1, wherein the photoswitchable protein is Dendra2, mOrange or PSCFP.

8. The method of claim 7, wherein photoswitching the protein comprises exposing a cancer cell or cells to blue light or ultraviolet light.

9. The method of claim 7, wherein the light has an approximate wavelength of 405 nm or 488 nm.

10. A kit for performing the method of claim 1 comprising two or more of the following:
(a) a plasmid encoding a photoswitchable protein;
(b) a cancer cell line;
(c) one or more imaging windows;
(d) an imaging box; and
(e) instructions for photoswitching, imaging of the cancer cells, and use of the imaging box.

11. The kit of claim 10, wherein the photoswitchable protein is Dendra2, mOrange or PSCFP.

12. The kit of claim 10, wherein the cancer cell line is a breast cancer, glioma, head and neck cancer, or melanoma cell line.

13. The kit of claim 12, wherein the cancer cell line is carcinoma, glioblastoma or melanoma.

14. A method for monitoring cell motility in a subject comprising:
(a) inserting cells transfected with a photoswitchable protein into a subject or obtaining a transgenic mouse subject expressing a photoswitchable protein;
(b) inserting an imaging window into the subject to allow observation of the cells;
(c) photoswitching the photoswitchable protein expressed in one or more cells; and
(d) observing the movement of the photo switched cell or cells over a period of time in the subject, thereby monitoring cell motility in the subject.

15. The method of claim 14, wherein the subject is a non-human animal model.

16. The method of claim 15, wherein the non-human animal model is a mouse or rat.

17. The method of claim 14, wherein the photoswitchable protein is Dendra2, mOrange or PSCFP.

18. The method of claim 17, wherein photoswitching the protein comprises exposing the transfected cell or cells to blue light or ultraviolet light.

19. The method of claim 17, wherein the light has an approximate wavelength of 405 nm or 488 nm.

20. The method of claim 14, wherein the cell is a cancer cell.

21. A kit for monitoring cell motility in a subject comprising two or more of the following:
(a) a plasmid encoding a photoswitchable protein;
(b) a cell line;
(c) one or more imaging windows;
(d) an imaging box; and
(e) instructions for photoswitching, imaging of the cells, and use of the imaging box.

22. (canceled)

23. A transgenic mouse expressing a photoswitchable protein.

24. (canceled)

25. The method of claim 1 for evaluating the efficacy of a potential cancer treatment for inhibiting metastasis in a subject comprising
observing the spreading of the photoswitched cell or cells over a period of time in the subject, wherein less spreading of the photoswitched cell or cells in the subject in comparison to a similar subject not having been administered the cancer treatment indicates that the cancer treatment is effective in inhibiting metastasis, or wherein movement of the photoswitched cell or cells in the subject that is not significantly less in comparison to a similar subject not having been administered the cancer treatment indicates that the cancer treatment is ineffective for inhibiting metastasis.
